(19) United States
(12) Patent Application Publication
(10) Pub. No.: US 2025/0222189 A1
(43) Pub. Date: Jul. 10, 2025

(54) Blood And Toxin Filter Device And Use Of Same

(71) Applicant: CYTOSORBENTS CORPORATION, Monmouth Junction, NJ (US)

(72) Inventors: Phillip P. CHAN, Cherry Hill, NJ (US); Vincent J. Capponi, Lawrenceville, NJ (US)

(21) Appl. No.: 17/917,322

(22) PCT Filed: Apr. 7, 2021
(86) PCT No.: PCT/US2021/026144

§ 371 Date: Feb. 14, 2023

Related U.S. Application Data
(60) Provisional application No. 63/006,191, filed on Apr. 7, 2020.

(51) Int. Cl. A61M 1/36; A61M 1/02; B01J 20/26; B01J 20/28

(52) U.S. Cl. CPC A61M 1/3679; A61M 1/0281; A61M 1/3618; B01J 20/267; B01J 20/2808; B01J 20/28083; B01J 20/28085; B01J 20/28092; A61M 2202/0014; A61M 2202/005

(57) ABSTRACT

The invention concerns devices and methods of filtering metallic nanoparticles and thrombi or microthrombi and other undesirable particles or molecules from blood or blood products.

### CROSS-REFERENCE TO RELATED APPLICATION

This application claims the benefit of U.S. Provisional Application No. 63/0006,191, filed on 7 Apr. 2020, the entire disclosure of which is incorporated herein by reference in its entirety.

### TECHNICAL FIELD

The invention concerns devices and methods for filtering particulates such as metallic nanoparticles and thrombi or microthrombi and sorbing other undesirable particles or molecules from blood or blood products.

### BACKGROUND

The invention is intended to meet the need to remove particulates such as metallic nanoparticles and thrombi or microthrombi and sorb other unwanted particles or molecules from blood or blood products and endogenously produced inflammatory mediators such as kinins, cytokines and complement associated with surgical trauma and infections. In some cases, the need results from surgical or other invasive procedures but also may have bacterial or viral origins. Treatments that involve the interaction of human blood and insoluble particulate material pose a hazard to the health of an already compromised patient

In other cases, such as with the COVID-19 pandemic, one of the observations is that patients may be hypercoagulable and suffering from disseminated intravascular thrombosis due to endothelial injury by a variety of inflammatory mediators, including activated complement. The endothelial surface gets damaged and due to a variety of interactions, including the coagulation factors such as von Willenbrand's factor and others, these small vessels will tend to clot. Circulating microthrombi may also be an issue. When these collect in the lung, they can contribute to ongoing lung injury. When these occur elsewhere, they can cause tissue ischemia.

There is a need in the art for improved treatments of such conditions and for efficient removal of metallic nanoparticles, inflammatory mediators and thrombi or microthrombi.

### SUMMARY OF THE INVENTION

In some aspects the invention concerns methods of filtering of particulates such as metallic nanoparticles and thrombi or microthrombi and sorbing other undesirable particles or molecules from blood or blood products, said method comprising: filtering said blood or blood products containing said metallic nanoparticles and thrombi or microthrombi with a filter element comprising cross-linked polymeric organic sorbent; and sorbing undesirable particles or molecules present in the blood or blood products into the cross-linked polymeric organic sorbent.

In some embodiments, the sorbent comprises cross-linked polymeric material derived from the reaction of a cross-linker with one or more of the following polymerizable monomers: divinyl-benzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, and methyl acrylate.

Some solid forms comprise particles having a diameter in the range of from about 0.1 microns to about 200 microns; and are characterized as having a pore structure having a total volume of pore sizes in the range of from 10 Å to 10,000 Å is greater than 0.5 cc/g to 3.0 cc/g dry polymer; wherein the ratio of pore volume between 10 Å to 3,000 Å in diameter to pore volume between 500 Å to 3,000 Å in diameter of the said cross-linked polymeric material is smaller than 7:1 and wherein the ratio of pore volume between 10 Å to 3,000 Å in diameter to pore volume between 10 Å to 6,000 Å in diameter of said cross-linked polymeric material is less than 2:1.

Undesirable molecules may include one or more of the following: biologically active molecules (BAMs), biological response modifiers (BRMs), products of hemolysis, products of membrane or cellular degradation, toxins, drugs, antibodies, prions and similar molecules found in stored blood and blood products.

Some undesirable particles or molecules may be metallic nanoparticles and thrombi or microthrombi. Other undesirable particles or molecules may be tissue or fatty matter released during a surgical or invasive procedure.

In some embodiments, the undesirable particles or molecules may be cellular debris, microbubbles, macromolecules, atherosclerotic plaque, atheroemboli, surgical debris, clumps of platelets, large protein aggregates like Fibrin or Von Willenbrands factor, Macrothrombi, thrombus, and the like.

Certain of the biologically active molecules may comprise inflammatory mediators, stimulators, or any combination thereof. Inflammatory mediators and stimulators may comprise cytokines, nitric oxide, thromboxanes, leukotrienes, platelet,-activating factor, prostaglandins, glycoproteins, kinins, kininogens, complement factors, cell-adhesion molecules, superantigens, monokines, chemokines, interferons, free radicals, proteases, arachidonic acid metabolites, prostacyclins, beta endorphins, myocardial depressant factors, anandimide, 2-arachadonylglycerol, tetrahydrobiopterin, serotonin, histamine, bradykinin, soluble CD40 ligand, bioactive lipids, oxidized lipids, hemoglobin, red cell particulates, membrane or cellular components, growth factors, glycoproteins, prions, toxins, endotoxins, drugs, vasoactive substances, foreign antigens, microvesicles, antibodies, or any combination thereof. In other embodiments the undesirable molecules comprise antibodies.

In some embodiments, the sorbent acts ex vivo. In other embodiments, the method is part of an extra corporeal treatment.

Some aspects of the invention concern blood purification devices comprising: (a) a device for contacting said blood or blood product that includes metallic nanoparticles and; and (b) a filtration device for removing metallic nanoparticles from said blood, said filtration device comprising a sorbent, said sorbent comprising primarily a plurality of solid forms comprising particles having a diameter in the range of from about 0.1 microns to about 200 microns; said sorbent comprising a cross-linked polymer; said sorbent being capable of sorbing non-metallic undesirable molecules. Sorbents may be any useful sorbent including those disclosed herein.

Some aspects of the invention concern blood purification devices comprising: (a) a device for contacting said blood with thrombi or microthrombi; and (b) a filtration device for removing thrombi or microthrombi from said blood, said filtration device comprising a sorbent, said sorbent comprising primarily a plurality of solid forms comprising particles having a diameter in the range of from about 0.1 microns to about 200 microns; said sorbent comprising a cross-linked polymer. Sorbents may be any useful sorbent including those disclosed herein.

The blood purification may further comprise a magnetic collection component for removing a portion of said metallic nanoparticles from the blood, the magnetic collection component disposed within the blood purification device such that blood is first contacted with the magnetic collection component prior to said blood contacting the filtration device. The metallic nanoparticles may comprise a coating capable of binding bacteria.

In certain embodiments, the filtration device comprises a cartridge containing the sorbent.

### BRIEF DESCRIPTION OF THE DRAWINGS

FIG. 1 depicts a circulation system according to the present invention.

### DETAILED DESCRIPTION OF ILLUSTRATIVE EMBODIMENTS

Some polymers comprise particles having a diameter in the range for 0.1 micron meters to 200 microns. Certain polymers are in the form of powder, beads or other regular or irregularly shaped particulate. The pore structure of some polymers is such that the total pore volume of pore size in the range of 50 \acute{Å} to 3000 \acute{Å} is greater than 0.5 cc/g to 3.0 cc/g dry polymer. In some embodiments, the polymer has a pore structure such that the total pore volume of pore size in the range of 50 \acute{Å} to 3000 \acute{Å} is greater than 0.5 cc/g to 3.0 cc/g dry polymer; wherein the ratio of pore volume between 50 \acute{Å} to 3,000 \acute{Å} (pore diameter) to pore volume between 500 \acute{Å} to 3,000 \acute{Å} (pore diameter) of the polymer is smaller than 200:1; and the ratio of pore volume between 50 \acute{Å} to 3,000 \acute{Å} (pore diameter) to pore volume between 1,000 \acute{Å} to 3,000 \acute{Å} (pore diameter) of the polymer is greater than 20:1.

In some embodiments, the polymer is a coated polymer comprising at least one crosslinking agent and at least one dispersing agent. The dispersing agents can be selected from such as hydroxyethyl cellulose, hydroxypopyl cellulose, poly(hydroxyethyl methacrylate), poly(hydroxyethyl acrylate), poly(hydroxypropyl methacrylate), poly(hydroxypropyl acrylate), poly(dimethylaminoethyl methacrylate), poly(dimethylaminoethyl acrylate), poly(diethylamimoethyl methacrylate), poly(diethylaminoethyl acrylate), poly(vinyl alcohol), poly(N-vinylpyrrolidinone), salts of poly(methacrylic acid), and salts of poly(acrylic acid) and mixtures thereof; the crosslinking agent selected from a group consisting of divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triiacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, divinylformamide and mixtures thereof; and the polymer is developed simultaneously with the formation of the coating, wherein the dispersing agent is chemically bound to the surface of the polymer.

Some preferred polymers comprise residues from one or more monomers selected from divnylbenzene and ethylvinylbezene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triiacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, divinylformamide and mixtures thereof.

Some suitable polymer include ion exchange polymers. In some embodiments, the polymer is a cellulosic polymer. In some embodiments, the polymers may be derivatized. Some polymers may be modified with an antibody or ligand. Such polymer may be porous or solid

Certain preferred polymers are porous highly crosslinked styrene or divinylbenzene copolymer. In some embodiments, the porous highly crosslinked styrene or divinylbenzene copolymer is a macroporous or mesoporous styrene-divinylbenzene-ethylstyrene copolymer subjected to a partial chloromethylation to a chlorine content of up to 7% molecular weight. In certain embodiments, the porous highly crosslinked styrene or divinylbenzene copolymer is a hypercrosslinked polystyrene produced from crosslinked styrene copolymers by an extensive chloromethylation and a subsequent post-crosslinking by treating with a Friedel-Crafts catalyst in a swollen state. In other embodiments, the porous highly crosslinked styrene or divinylbenzene copolymer is a hypercrosslinked polystyrene produced from crosslinked styrene copolymers by an extensive additional post-crosslinking in a swollen state with bifunctional crosslinking agents selected from the group comprising of monochlorodimethyl ether and p-xylilene dichloride.

In another aspect of the invention, the polymer is a hydrophilic self-wetting polymer that can be administered as dry powder or dry particulate containing hydrophilic functional groups such as chlorine, amines, hydroxyl, sulfonate, and carboxyl groups Certain polymer may be pyrolyzed.

In some embodiments, the polymer materials used as the sorbent are substantially not metabolizable by human and animal. Certain polymers may be irregular or regular shaped particulates such as powders, beads, or other forms with a diameter in the range of 0.1 microns to 200 microns

The polymers used in the instant invention preferably have a biocompatible and hemocompatible exterior surface coatings but are not absolutely necessary, especially in certain circumstances, such as oral or rectal administration. Certain of these coatings are covalently bound to the polymer particle (beads, for example) by free-radical grafting. The free-radical grafting may occur, for example, during the transformation of the monomer droplets into polymer beads. The dispersant coating and stabilizing the monomer droplets becomes covalently bound to the droplet surface as the monomers within the droplets polymerize and are converted into polymer. Biocompatible and hemocompatible exterior surface coatings can be covalently grafted onto the preformed polymer beads if the dispersant used in the suspension polymerization is not one that imparts biocompatibility or hemocompatibility. Grafting of biocompatible and hemocompatible coatings onto preformed polymer beads is carried out by activating free-radical initiators in the presence of either the monomers or low molecular weight oligomers of the polymers that impart biocompatibility or hemocompatibility to the surface coating.

By “biocompatible”, it is meant that the polymer is capable of contact with living tissues or organisms without causing harm during the time that the polymer is in contact with the tissue or organism. In some embodiments, it is intended that the polymer is tolerated by the gut and alimentary canal of the organism. The polymers of the present invention are preferably non-toxic.

In some embodiments, the polymer has a preferential pore structure such that the total pore volume of pore size in the range of 50 \acute{Å} to 3000 \acute{Å} is greater than 0.5 cc/g to 3.0 cc/g dry polymer; wherein the ratio of pore volume between 50 \acute{Å} to 3,000 \acute{Å} (pore diameter) to the pore volume between 500 \acute{Å} to 3,000 \acute{Å} (pore diameter) of the polymer is smaller than 200:1; and the ratio of pore volume between 50 \acute{Å} to 3,000 \acute{Å} in diameter to the pore volume between 1,000 \acute{Å} to 3,000 \acute{Å} in diameter of the polymer is greater than 20:1. The said ratios can be alternatively specified in terms of pore surface area (such as the ratio of pore surface area between 50 \acute{Å} to 3,000 \acute{Å} to pore surface area between 500 \acute{Å} to 3,000 \acute{Å} of the polymer); and therefore is an alternative way of specifying the same pore structure.

In certain embodiments, the sorbent has a pore structure having a total volume of pore sizes in the range of from 10 Å to 10,000 Å is greater than 0.5 cc/g to 3.0 cc/g dry polymer; wherein the ratio of pore volume between 10 Å to 3,000 Å in diameter to pore volume between 500 Å to 3,000 Å in diameter of the said cross-linked polymeric material is smaller than 7:1 and wherein the ratio of pore volume between 10 Å to 3,000 Å in diameter to pore volume between 10 Å to 6,000 Å in diameter of said cross-linked polymeric material is less than 2:1.

In some embodiments, the sorbent has:
a) a pore structure wherein at least ⅓ of the pore volume in pores having diameters between 50 Å and 40,000 Å is in pores having diameters between 100 Å and 1,000 Å; or
(b) a pore structure wherein at least ½ of the pore volume in pores having diameters between 50 Å and 40,000 Å is in pores having diameters between 1000 Å and 10,000 Å; or
(c) a pore structure wherein at least ⅓ of the pore volume in pores having diameters between 50 Å and 40,000 Å is in pores having diameters between 10,000 Å and 40,000 Å.

Suitable crosslinking agents include divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triiacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, divinylformamide and mixtures thereof. Preferably, the polymer is developed simultaneously with the formation of the coating, such that the dispersing agent gets chemically bound to the surface of the polymer.

Preferred polymers include those derived from one or more monomers selected from divnylbenzene and ethylvinylbezene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triiacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, divinylformamide and mixtures thereof.

Some preferred polymers are ion exchange polymers.

Some preferred polymers are cellulosic polymers. Suitable polymers include cross-linked dextran gels such as Sephadex®.

Certain preferred polymers are porous highly crosslinked styrene or divinylbenzene copolymer. Some of these polymers are a macroporous or mesoporous styrene-divinylbenzene-ethylstyrene copolymer subjected to a partial chloromethylation to a chlorine content of up to 7% molecular weight. Other of these polymers are a hypercrosslinked polystyrene produced from crosslinked styrene copolymers by an extensive chloromethylation and a subsequent post-crosslinking by treating with a Friedel-Crafts catalyst in a swollen state. Yet other of these polymers are a hypercrosslinked polystyrene produced from crosslinked styrene copolymers by an extensive additional post-crosslinking in a swollen state with bifunctional crosslinking agents selected from the group comprising of monochlorodimethyl ether and p-xylilene dichloride

Some polymers useful in the practice of the invention are hydrophilic self wetting polymers that can be administered as dry powder containing hydrophilic functional groups such as, amines, hydroxyl, sulfonate, and carboxyl groups.

Certain polymers useful in the invention are macroporous polymers prepared from the polymerizable monomers of styrene, divinylbenzene, ethylvinylbenzene, and the acrylate and methacrylate monomers such as those listed below by manufacturer. Rohm and Haas Company, (now part of Dow Chemical Company): (i) macroporous polymeric sorbents such as Amberlite™ XAD-1, Amberlite™ XAD-2, Amberlite™ XAD-4, Amberlite™ XAD-7, Amberlite™ XAD-7HP, Amberlite™ XAD-8, Amberlite™ XAD-16, Amberlite™ XAD-16 HP, Amberlite™ XAD-18, Amberlite™ XAD-200, Amberlite™ XAD-1180, Amberlite™ XAD-2000, Amberlite™ XAD-2005, Amberlite™ XAD-2010, Amberlite™ XAD-761, and Amberlite™ XE-305, and chromatographic grade sorbents such as Amberchrom™ CG 71,s,m,c, Amberchrom™ CG 161,s,m,c, Amberchrom™ CG 300,s,m,c, and Amberchrom™ CG 1000,s,m,c. Dow Chemical Company: Dowex® Optipore™ L-493, Dowex® Optipore™ V-493, Dowex® Optipore™ V-502, Dowex® Optipore™ L-285, Dowex® Optipore™ L-323, and Dowex® Optipore™ V-503. Lanxess (formerly Bayer and Sybron): Lewatit® VPOC 1064 MD PH, Lewatit® VPOC 1163, Lewatit® OC EP 63, Lewatit® S 6328A, Lewatit® OC 1066, and Lewatit® 60/150 MIBK. Mitsubishi Chemical Corporation: Diaion® HP 10, Diaion® HP 20, Diaion® HP 21, Diaion® HP 30, Diaion® HP 40, Diaion® HP 50, Diaion® SP70, Diaion® SP 205, Diaion® SP 206, Diaion® SP 207, Diaion® SP 700, Diaion® SP 800, Diaion® SP 825, Diaion® SP 850, Diaion® SP 875, Diaion® HP 1 MG, Diaion® HP 2 MG, Diaion® CHP 55A, Diaion® CHP 55Y, Diaion® CHP 20A, Diaion® CHP 20Y, Diaion® CHP 2MGY, Diaion® CHP 20P, Diaion® HP 20SS, Diaion® SP 20SS, and Diaion® SP 207SS. Purolite Company: Purosorb™ AP 250 and Purosorb™ AP 400.

In some embodiments, the metallic nanoparticles comprise a pure metal such as gold, platinum, silver, titanium, zinc, cerium, iron, and thallium. In some embodiments, the metallic nanoparticle comprises gold. In some embodiments, the metallic nanoparticle comprises platinum. In some embodiments, the metallic nanoparticle comprises silver. In some embodiments, the metallic nanoparticle comprises titanium. In some embodiments, the metallic nanoparticle comprises zinc. In some embodiments, the metallic nanoparticle comprises cerium. In some embodiments, the metallic nanoparticle comprises iron. In some embodiments, the metallic nanoparticle comprises thallium.

In some embodiments, the metallic nanoparticles comprise a compound of a pure metal such as oxides, hydroxides, sulfides, phosphates, fluorides, and chlorides of pure metals. In some embodiments, the metallic nanoparticle comprises an oxide of a pure metal. In some embodiments, the metallic nanoparticle comprises a hydroxide of a pure metal. In some embodiments, the metallic nanoparticle comprises a sulfide of a pure metal. In some embodiments, the metallic nanoparticle comprises a phosphate of a pure metal. In some embodiments, the metallic nanoparticle comprises a fluoride of a pure metal. In some embodiments, the metallic nanoparticle comprises a chloride of a pure metal.

In some embodiments, the metallic nanoparticles have a size in the range of from about 1 nm to about 100 nm. In some embodiments, the metallic nanoparticles have a size in the range of from about 10 nm to about 90 nm. In some embodiments, the metallic nanoparticles have a size in the range of from about 20 nm to about 80 nm. In some embodiments, the metallic nanoparticles have a size in the range of from about 30 nm to about 70 nm. In some embodiments, the metallic nanoparticles have a size in the range of from about 40 nm to about 60 nm. In some embodiments, the metallic nanoparticles have a size in the range of from about 45 nm to about 55 nm.

In some embodiments, the metallic nanoparticles have a size in the range of from about 1 nm to about 5 nm; or from about 5 nm to about 10 nm; or from about 10 nm to about 15 nm; or from about 15 nm to about 20 nm; or from about 20 nm to about 25 nm; or from about 25 nm to about 30 nm; or from about 30 nm to about 35 nm; or from about 35 nm to about 40 nm; or from about 40 nm to about 45 nm; or from about 45 nm to about 50 nm; or from about 50 nm to about 55 nm; or from about 55 nm to about 60 nm; or from about 60 nm to about 65 nm; or from about 65 nm to about 70 nm; or from about 70 nm to about 75 nm; or from about 75 nm to about 80 nm; or from about 80 nm to about 85 nm; or from about 85 nm to about 90 nm; or from about 90 nm to about 95 nm; or from about 95 nm to about 100 nm.

In some embodiments, the thrombi or microthrombi have a size in the range of from about 0.5 μm to about 100 μm. In some embodiments, the thrombi or microthrombi have a size in the range of from about 1 μm to about 90 μm. In some embodiments, the thrombi or microthrombi have a size in the range of from about 5 μm to about 80 μm. In some embodiments, the thrombi or microthrombi have a size in the range of from about 10 μm to about 70 μm. In some embodiments, the thrombi or microthrombi have a size in the range of from about 15 μm to about 65 μm. In some embodiments, the thrombi or microthrombi have a size in the range of from about 20 μm to about 60 μm. In some embodiments, the thrombi or microthrombi have a size in the range of from about 25 μm to about 55 μm. In some embodiments, the thrombi or microthrombi have a size in the range of from about 30 μm to about 50 μm. In some embodiments, the thrombi or microthrombi have a size in the range of from about 35 μm to about 45 μm. In some embodiments, the thrombi or microthrombi have a size of about 40 μm.

In some embodiments, the thrombi or microthrombi have a size in the range of from about 0.5 μm to about 5 μm; or from about 5 μm to about 10 μm; or from about 10 μm to about 15 μm; or from about 15 μm to about 20 μm; or from about 20 μm to about 25 μm; or from about 25 μm to about 30 μm; or from about 30 μm to about 35 μm; or from about 35 μm to about 40 μm; or from about 40 μm to about 45 μm; or from about 45 μm to about 50 μm; or from about 50 μm to about 55 μm; or from about 55 μm to about 60 μm; or from about 60 μm to about 65 μm; or from about 65 μm to about 70 μm; or from about 70 μm to about 75 μm; or from about 75 μm to about 80 μm; or from about 80 μm to about 85 μm; or from about 85 μm to about 90 μm; or from about 90 μm to about 95 μm; or from about 95 μm to about 100 μm.

In some embodiments, the thrombi or microthrombi have a size in the range of from about 30 μm to about 70 μm. In some embodiments, the thrombi or microthrombi have a size in the range of from about 30 μm to about 35 μm; or from about 35 μm to about 40 μm; or from about 40 μm to about 45 μm; or from about 45 μm to about 50 μm; or from about 50 μm to about 55 μm; or from about 55 μm to about 60 μm; or from about 60 μm to about 65 μm; or from about 65 μm to about 70 μm.

In some embodiments, the thrombi or microthrombi have a size in the range of from about 0.5 μm to about 15 μm. In some embodiments, the thrombi or microthrombi have a size in the range of from about 0.75 μm to about 10 μm. In some embodiments, the thrombi or microthrombi have a size in the range of from about 1 μm to about 5 μm. In some embodiments, the thrombi or microthrombi have a size in the range of from about 1.5 μm to about 3 μm. In some embodiments, the thrombi or microthrombi have a size of about 2 μm.

In some embodiments, the thrombi or microthrombi have a size in the range of from about 0.5 μm to about 1 μm; or from about 1 μm to about 1.5 μm; or from about 1.5 μm to about 2 μm; or from about 2 μm to about 2.5 μm; or from about 2.5 μm to about 3 μm; or from about 3 μm to about 3.5 μm; or from about 3.5 μm to about 4 μm; or from about 4 μm to about 4.5 μm; or from about 4.5 μm to about 5 μm; or from about 5 μm to about 5.5 μm; or from about 5.5 μm to about 6 μm; or from about 6 μm to about 6.5 μm; or from about 6.5 μm to about 7 μm; or from about 7 μm to about 7.5 μm; or from about 7.5 μm to about 8 μm; or from about 8 μm to about 8.5 μm; or from about 8.5 μm to about 9 μm; or from about 9 μm to about 9.5 μm; or from about 9.5 μm to about 10 μm; or from about 10 μm to about 10.5 μm; or from about 10.5 μm to about 11 μm; or from about 11 μm to about 11.5 μm; or from about 11.5 μm to about 12 μm; or from about 12 μm to about 12.5 μm; or from about 12.5 μm to about 13 μm; or from about 13 μm to about 13.5 μm; or from about 13.5 μm to about 14 μm; or from about 14 μm to about 14.5 μm; or from about 14.5 μm to about 15 μm.

As used herein, the term “sorbent” includes adsorbents and absorbents.

As used herein, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The phrase “residue” or “monomer residue” refers to the portion of a monomer that is incorporated into a polymer when the monomer is polymerized. For example, when R-x is reacted with R′-y to produce R—R′ with x and y being freed during the reaction, R and R′ are monomer residues.

In some embodiments, provided are methods of filtering thrombi or metallic nanoparticles and other undesirable particles or molecules from blood or blood products, said method comprising:
filtering said blood or blood products containing said thrombi or metallic nanoparticles with a filter element comprising a cross-linked polymeric organic sorbent to remove the thrombi or metallic nanoparticles; and
sorbing undesirable particles or molecules present in the blood or blood products into the cross-linked polymeric organic sorbent.

In some embodiments, provided are blood purification devices comprising:
(a) a device for contacting said blood with thrombi or metallic nanoparticles; and
(b) a filtration device for removing thrombi or metallic nanoparticles from said blood, said filtration device comprising a sorbent, said sorbent comprising primarily a plurality of solid forms comprising particles having a diameter in the range of from about 0.1 microns to about 200 microns; said sorbent comprising a cross-linked polymer; said sorbent being capable of sorbing non-metallic undesirable molecules.

In some embodiments, provided are methods of filtering thrombi or metallic nanoparticles from blood or blood products, said method comprising:

filtering said blood or blood products containing said thrombi or metallic nanoparticles with a filter element comprising a cross-linked polymeric organic sorbent to remove the thrombi or metallic nanoparticles.

In some embodiments, the sorbent comprises cross-linked polymeric material derived from the reaction of a cross-linker with one or more of the following polymerizable monomers: divinyl-benzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, and methyl acrylate.

In some embodiments, the sorbent comprises said solid forms which comprise particles having a diameter in the range of from about 0.1 microns to about 200 microns; and are characterized as having a pore structure having a total volume of pore sizes in the range of from 10 Å to 10,000 Å is greater than 0.5 cc/g to 3.0 cc/g dry polymer; wherein the ratio of pore volume between 10 Å to 3,000 Å in diameter to pore volume between 500 Å to 3,000 Å in diameter of the said cross-linked polymeric material is smaller than 7:1 and wherein the ratio of pore volume between 10 Å to 3,000 Å in diameter to pore volume between 10 Å to 6,000 Å in diameter of said cross-linked polymeric material is less than 2:1.

In some embodiments, the undesirable particles or molecules comprise one or more of the following: biologically active molecules (BAMs), biological response modifiers (BRMs), products of hemolysis, products of membrane or cellular degradation, toxins, drugs, antibodies, prions and similar molecules found in stored blood and blood products.

In some embodiments, the biologically active molecules comprise (i) inflammatory mediators, (ii) stimulators, (iii) microthrombi, (iv) tissue or fatty matter released during a surgical or invasive procedure, or (v) any combination thereof.

In some embodiments, the inflammatory mediators and stimulators comprise cytokines, nitric oxide, thromboxanes, leukotrienes, platelet,-activating factor, prostaglandins, glycoproteins, kinins, kininogens, complement factors, cell-adhesion molecules, superantigens, monokines, chemokines, interferons, free radicals, proteases, arachidonic acid metabolites, prostacyclins, beta endorphins, myocardial depressant factors, anandimide, 2-arachadonylglycerol, tetrahydrobiopterin, serotonin, histamine, bradykinin, soluble CD40 ligand, bioactive lipids, oxidized lipids, hemoglobin, red cell particulates, membrane or cellular components, growth factors, glycoproteins, prions, toxins, endotoxins, drugs, vasoactive substances, foreign antigens, microvesicles, antibodies, or any combination thereof.

In some embodiments, the sorbent acts ex vivo. In some embodiments, the method is part of an extra corporeal treatment.

In some embodiments, the thrombi is viral-induced thrombi. In some embodiments, the thrombi is bacteria-induced thrombi.

In some embodiments, the thrombi have a size in a range of from about 0.5 μm to about 100 μm. In some embodiments, the thrombi have a size in a range of from about 1 μm to about 90 μm. In some embodiments, the thrombi have a size in a range of from about 5 μm to about 80 μm. In some embodiments, the thrombi have a size in a range of from about 10 μm to about 70 μm. In some embodiments, the thrombi have a size in a range of from about 15 μm to about 65 μm. In some embodiments, the thrombi have a size in a range of from about 20 μm to about 60 μm. In some embodiments, the thrombi have a size in a range of from about 30 μm to about 50 μm.

In some embodiments, the thrombi have a size in a range of from about 0.5 μm to about 5 μm; or from about 5 μm to about 10 μm; or from about 10 μm to about 15 μm; or from about 15 μm to about 20 μm; or from about 20 μm to about 25 μm; or from about 25 μm to about 30 μm; or from about 30 μm to about 35 μm; or from about 35 μm to about 40 μm; or from about 40 μm to about 45 μm; or from about 45 μm to about 50 μm; or from about 50 μm to about 55 μm; or from about 55 μm to about 60 μm; or from about 60 μm to about 65 μm; or from about 65 μm to about 70 μm; or from about 70 μm to about 75 μm; or from about 75 μm to about 80 μm; or from about 80 μm to about 85 μm; or from about 85 μm to about 90 μm; or from about 90 μm to about 95 μm; or from about 95 μm to about 100 μm.

In some embodiments, the thrombi have a size in a range of from about 0.5 μm to about 15 μm. In some embodiments, the thrombi have a size in a range of from about 0.75 μm to about 10 μm. In some embodiments, the thrombi have a size in a range of from about 1 μm to about 5 μm. In some embodiments, the thrombi have a size in a range of from about 1.5 μm to about 3 μm. In some embodiments, the thrombi have a size of about 2 μm.

In some embodiments, the blood purification device further comprises a magnetic collection component for removing a portion of said metallic nanoparticles from the blood, the magnetic collection component disposed within the blood purification device such that blood is first contacted with the magnetic collection component prior to said blood contacting the filtration device. In some embodiments, filtration device comprises a cartridge containing said sorbent.

In some embodiments, the undesirable particles or molecules comprise antibodies. In some embodiments, the metallic nanoparticles comprise a coating capable of binding bacteria. In some embodiments, antibodies are attached to the metallic nanoparticles.

The following Examples are provided to illustrate some of the concepts described within this disclosure. While the Examples are considered to provide an embodiment, it should not be considered to limit the more general embodiments described herein.

### Example 1

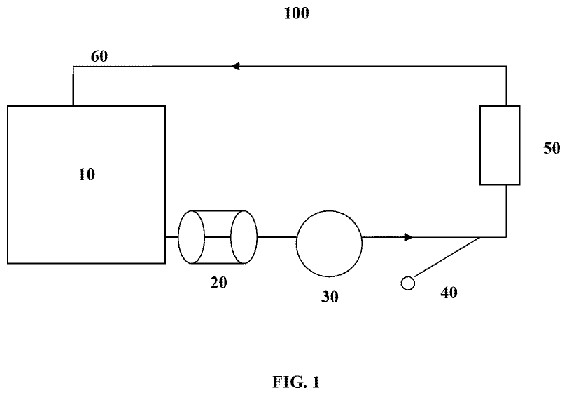

The circulation system used is depicted in FIG. 1, whereby circulation system 100 includes a reservoir 10 holding a circulating fluid, such as water, blood or blood product. The reservoir holdings are then circulated by pump 30 and passed through filter 20 to remove contaminants. Pump 30 ensures the circulating fluid is circulated throughout circulation system 100. Particulate solution is injected into circulation system 100 via the injection port 40 which includes a Y-connector configuration. The particulate solution is passed through device 50 that includes the sorbents described herein. The circulation system 100 includes an outlet 60 for collection of material to be tested for particulate concentration.

The materials used include the following:
Blood Sets, Henry Schein Part #6669063,
Slip Luer Syringes, National Scientific Part #S7510-10,
Low Flow Pump, Fisher/Control Company Part #3386,
Peristaltic Pump,
NaCl, Sigma Part #S1679,
Purified Water,
2 μm Particle Size Standard, Thermo Count-Cal Part CC02, Lot 41740, and
HIAC Liquid Particle Counting System, HIAC Model #9307.

### Solution Preparation-0.9% Saline Solution (4450 ml)

4380 ml [(4300 ml human volume)+80 ml (for blank sampling)] of 0.9% saline solution was prepared by adding 39.42 g of NaCl to 4380 ml of purified water. The solution was stirred until all the NaCl was dissolved. The osmolality of the solution was determined to be 290±10 mOsm.

### Solution Preparation—2 μm Standard

50 ml of particle size standard solution was diluted to 100 ml and 57 ml of the standard solution was required for a run of 5 hours at 0.19 ml/min. The particle count was as follows:

$$3000 ( \pm 10 \% ) particles / ml * 50 ml = 150 , 000 ( \pm 10 \% ) particles total$$

$$150000 ( \pm 10 \% ) particles / 100 ml = 1500 ( \pm 150 ) particles / ml$$

### Procedure

The flow rate for pump 30 is verified to be within 10% of the displayed values.

Polycarbonate bottles are blanked by analyzing the samples on the particulate counting system (three 5 ml aliquots, disregarding the first aliquot). For each vial, a summary report of particle count per volume, including the count specifically at 2 μm, was generated, as was a full summary for all glassware and samples.

The glassware blank limits for particles≥10 μm the total cumulative count should be less than 10 and the glassware blank limits for particles≥25 μm the total count should be less than 2.

The loop system 100 is blanked with a 0.22 μm filter 20 and a CytoSorb device 50 for at least 30 minutes at a flow rate>200 ml/min. About 40 ml of solution into a blanked polycarbonate bottle was collected. This is the loop blank.

If the loop blank passes the particulate glassware requirements, the 0.22 μm filter 20 is removed and the system is allowed to run at about 20 ml/min for a few minutes. About 50 ml of solution into a blanked particulate vial was collected. This is the baseline sample.

About 40 ml of particulate sample into a blanked particulate vial was collected.

About 5.7 ml of particulate solution was injected into the y-connector injection port 40 of the system 100 with a large bore syringe. The timer began after the solution was injected.

About 40 ml of solution was collected from the outlet 60 every 15 minutes into the blanked particulate vials, and another about 5.7 ml of particulate solution was injected into the y-connector injection port 40 every 30 minutes for the duration of the experiment (5 hours).

The baseline, particulate solution and all of the outlet samples for particulate content using the full summary report was then analyzed.

### Results

The particulate content of the outlet, in addition to the particulate content of blanked polycarbonate vials, recirculation system and particulate solution used for injections, are summarized below in Table 1.

TABLE 1
Experimental Particulate Content Measurements

| | | | | | Corrected |
|---|---|---|---|---|---|
| Vial | Desc | Counts | Blanked Vial | Baseline | Count |
| 50 | Baseline | 3.1 | 5 | N/A | 0 |
| 4 | Particulate | 668.8 | 1.7 | 0 | 667.1 |
| | Solution | | | | |
| 51 | 15 | 10.3 | 8.8 | 0 | 1.5 |
| 46* | 30 | 5.8 | 3 | 0 | 2.8 |
| 24 | 45 | 6.3 | 1.8 | 0 | 4.5 |
| 78* | 60 | 8.6 | 4.5 | 0 | 4.1 |
| 32 | 75 | 16.7 | 10.9 | 0 | 5.8 |
| 34* | 90 | 8.4 | 3 | 0 | 5.4 |
| 53 | 105 | 7.2 | 2.5 | 0 | 4.7 |
| 56* | 120 | 4.2 | 2.1 | 0 | 2.1 |
| 64 | 135 | 5.3 | 3 | 0 | 2.3 |
| 10* | 150 | 5.4 | 1.8 | 0 | 3.6 |
| 75 | 165 | 4.6 | 4.2 | 0 | 0.4 |
| 47* | 180 | 6.4 | 5.2 | 0 | 1.2 |
| 61 | 195 | 7.5 | 1 | 0 | 6.5 |
| 76* | 210 | 6.3 | 2 | 0 | 4.3 |
| 25 | 225 | 6 | 1 | 0 | 5 |
| 77* | 240 | 5.2 | 0.4 | 0 | 4.8 |
| 21 | 255 | 4.4 | 1.2 | 0 | 3.2 |
| 41* | 270 | 11.5 | 7.4 | 0 | 4.1 |
| 62 | 285 | 13.9 | 7.8 | 0 | 6.1 |
| 48 | 300 | 5 | 1.9 | 0 | 3.1 |

*The vials denoted with an asterisk mark represent the time points where beads (667.1/ml, or 3800 particles total) were injected. The subsequent time points are when the particulates would have been present in the outlet port of the recirculation system, e.g., particles injected at 120 minutes would have been present in the outlet at 135 minutes.

### CONCLUSION

A CytoSorb device 50 was challenged with removing more than 38,000 2 μm beads over the course of 5 hours and removed the vast majority of particles passing through the system.

Coupled with the documented hemocompatibility and cytokine filtration, CytoSorb proved its effectiveness at removing small particulate matter and its ability to be functionalized as a depth filter for treatments that may have concerns about particulate matter.

What is claimed:

1. A method of filtering thrombi or metallic nanoparticles and undesirable particles or molecules from blood or blood products, said method comprising:
filtering said blood or blood products containing said thrombi or metallic nanoparticles with a filter element comprising a cross-linked polymeric organic sorbent to remove the thrombi or metallic nanoparticles; and
sorbing undesirable particles or molecules present in the blood or blood products into the cross-linked polymeric organic sorbent.

2. The method of claim 1, wherein said sorbent comprises cross-linked polymeric material derived from the reaction of a cross-linker with one or more of the following polymerizable monomers: divinyl-benzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, and methyl acrylate.

3. The method of claim 1 or 2, wherein said solid forms comprise particles having a diameter in the range of from about 0.1 microns to about 200 microns; and are characterized as having a pore structure having a total volume of pore sizes in the range of from 10 Å to 10,000 Å is greater than 0.5 cc/g to 3.0 cc/g dry polymer; wherein the ratio of pore volume between 10 Å to 3,000 Å in diameter to pore volume between 500 Å to 3,000 Å in diameter of the said cross-linked polymeric material is smaller than 7:1 and wherein the ratio of pore volume between 10 Å to 3,000 Å in diameter to pore volume between 10 Å to 6,000 Å in diameter of said cross-linked polymeric material is less than 2:1.

4. The method of any one of claims 1-3, wherein said undesirable particles or molecules comprise one or more of the following: biologically active molecules (BAMs), biological response modifiers (BRMs), products of hemolysis, products of membrane or cellular degradation, toxins, drugs, antibodies, prions and similar molecules found in stored blood and blood products.

5. The method of claim 4, wherein the biologically active molecules comprise (i) inflammatory mediators, (ii) stimulators, (iii) microthrombi, (iv) tissue or fatty matter released during a surgical or invasive procedure, or (v) any combination thereof.

6. The method of claim 5, wherein said inflammatory mediators and stimulators comprise cytokines, nitric oxide, thromboxanes, leukotrienes, platelet,-activating factor, prostaglandins, glycoproteins, kinins, kininogens, complement factors, cell-adhesion molecules, superantigens, monokines, chemokines, interferons, free radicals, proteases, arachidonic acid metabolites, prostacyclins, beta endorphins, myocardial depressant factors, anandimide, 2-arachadonylglycerol, tetrahydrobiopterin, serotonin, histamine, bradykinin, soluble CD40 ligand, bioactive lipids, oxidized lipids, hemoglobin, red cell particulates, membrane or cellular components, growth factors, glycoproteins, prions, toxins, endotoxins, drugs, vasoactive substances, foreign antigens, microvesicles, antibodies, or any combination thereof.

7. The method of claim 1 wherein the undesirable particles or molecules comprise antibodies.

8. The method of any of claims 1-7, wherein the sorbent acts ex vivo.

9. The method of any of claims 1-8, wherein the method is part of an extra corporeal treatment.

10. The method of any of claims 1-9, wherein the thrombi is viral-induced thrombi.

11. The method of any of claims 1-9, wherein the thrombi is bacteria-induced thrombi.

12. The method of claim 1, wherein the thrombi have a size in a range of from about 0.5 μm to about 100 μm.

13. The method of claim 1, wherein the thrombi have a size in a range of from about 1 μm to about 90 μm.

14. The method of claim 1, wherein the thrombi have a size in a range of from about 5 μm to about 80 μm.

15. The method of claim 1, wherein the thrombi have a size in a range of from about 10 μm to about 70 μm.

16. The method of claim 1, wherein the thrombi have a size in a range of from about 15 μm to about 65 μm.

17. The method of claim 1, wherein the thrombi have a size in a range of from about 20 μm to about 60 μm.

18. The method of claim 1, wherein the thrombi have a size in a range of from about 30 μm to about 50 μm.

19. The method of claim 1, wherein the thrombi have a size in a range of from about 0.5 μm to about 5 μm; or from about 5 μm to about 10 μm; or from about 10 μm to about 15 μm; or from about 15 μm to about 20 μm; or from about 20 μm to about 25 μm; or from about 25 μm to about 30 μm; or from about 30 μm to about 35 μm; or from about 35 μm to about 40 μm; or from about 40 μm to about 45 μm; or from about 45 μm to about 50 μm; or from about 50 μm to about 55 μm; or from about 55 μm to about 60 μm; or from about 60 μm to about 65 μm; or from about 65 μm to about 70 μm; or from about 70 μm to about 75 μm; or from about 75 μm to about 80 μm; or from about 80 μm to about 85 μm; or from about 85 μm to about 90 μm; or from about 90 μm to about 95 μm; or from about 95 μm to about 100 μm.

20. The method of claim 1, wherein the thrombi have a size in a range of from about 0.5 μm to about 15 μm.

21. The method of claim 1, wherein the thrombi have a size in a range of from about 0.75 μm to about 10 μm.

22. The method of claim 1, wherein the thrombi have a size in a range of from about 1 μm to about 5 μm.

23. The method of claim 1, wherein the thrombi have a size in a range of from about 1.5 μm to about 3 μm.

24. The method of claim 1, wherein the thrombi have a size of about 2 μm.

25. A blood purification device comprising:
(a) a device for contacting said blood with thrombi or metallic nanoparticles; and
(b) a filtration device for removing thrombi or metallic nanoparticles from said blood, said filtration device comprising a sorbent, said sorbent comprising primarily a plurality of solid forms comprising particles having a diameter in the range of from about 0.1 microns to about 200 microns; said sorbent comprising a cross-linked polymer; said sorbent being capable of sorbing non-metallic undesirable molecules.

26. The blood purification device of claim 25, further comprising a magnetic collection component for removing a portion of said metallic nanoparticles from the blood, the magnetic collection component disposed within the blood purification device such that blood is first contacted with the magnetic collection component prior to said blood contacting the filtration device.

27. The blood purification device of claim 25 or 26, wherein said metallic nanoparticles comprise a coating capable of binding bacteria.

28. The blood purification device of any one of claims 25-27, wherein said sorbent comprises cross-linked polymeric material derived from the reaction of a cross-linker with one or more of the following polymerizable monomers: divinyl-benzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, and methyl acrylate.

29. The blood purification device of any one of claims 25-28, wherein said solid form is characterized as having a pore structure having a total volume of pore sizes in the range of from 10 Å to 10,000 Å is greater than 0.5 cc/g to 3.0 cc/g dry polymer; wherein the ratio of pore volume between 10 Å to 3,000 Å in diameter to pore volume between 500 Å to 3,000 Å in diameter of the said cross-linked polymeric material is smaller than 7:1 and wherein the ratio of pore volume between 10 Å to 3,000 Å in diameter to pore volume between 10 Å to 6,000 Å in diameter of said cross-linked polymeric material is less than 2:1.

30. The blood purification device of any one of claims 25-29, wherein said filtration device comprises a cartridge containing said sorbent.

31. The blood purification device of any of claims 25-30, wherein the thrombi is viral-induced thrombi.

32. The blood purification device of any of claims 25-30, wherein the thrombi is bacteria-induced thrombi.

33. The blood purification device of claim 25, wherein the thrombi have a size in a range of from about 0.5 μm to about 100 μm.

34. The blood purification device of claim 25, wherein the thrombi have a size in a range of from about 1 μm to about 90 μm.

35. The blood purification device of claim 25, wherein the thrombi have a size in a range of from about 5 μm to about 80 μm.

36. The blood purification device of claim 25, wherein the thrombi have a size in a range of from about 10 μm to about 70 μm.

37. The blood purification device of claim 25, wherein the thrombi have a size in a range of from about 15 μm to about 65 μm.

38. The blood purification device of claim 25, wherein the thrombi have a size in a range of from about 20 μm to about 60 μm.

39. The blood purification device of claim 25, wherein the thrombi have a size in a range of from about 30 μm to about 50 μm.

40. The blood purification device of claim 25, wherein the thrombi have a size in a range of from about 0.5 μm to about 5 μm; or from about 5 μm to about 10 μm; or from about 10 μm to about 15 μm; or from about 15 μm to about 20 μm; or from about 20 μm to about 25 μm; or from about 25 μm to about 30 μm; or from about 30 μm to about 35 μm; or from about 35 μm to about 40 μm; or from about 40 μm to about 45 μm; or from about 45 μm to about 50 μm; or from about 50 μm to about 55 μm; or from about 55 μm to about 60 μm; or from about 60 μm to about 65 μm; or from about 65 μm to about 70 μm; or from about 70 μm to about 75 μm; or from about 75 μm to about 80 μm; or from about 80 μm to about 85 μm; or from about 85 μm to about 90 μm; or from about 90 μm to about 95 μm; or from about 95 μm to about 100 μm.

41. The blood purification device of claim 25, wherein the thrombi have a size in a range of from about 0.5 μm to about 15 μm.

42. The blood purification device of claim 25, wherein the thrombi have a size in a range of from about 0.75 μm to about 10 μm.

43. The blood purification device of claim 25, wherein the thrombi have a size in a range of from about 1 μm to about 5 μm.

44. The blood purification device of claim 25, wherein the thrombi have a size in a range of from about 1.5 μm to about 3 μm.

45. The blood purification device of claim 25, wherein the thrombi have a size of about 2 μm.

46. A method of filtering thrombi or metallic nanoparticles from blood or blood products, said method comprising:
filtering said blood or blood products containing said thrombi or metallic nanoparticles with a filter element comprising a cross-linked polymeric organic sorbent to remove the thrombi or metallic nanoparticles.

47. The method of claim 46, wherein said sorbent comprises cross-linked polymeric material derived from the reaction of a cross-linker with one or more of the following polymerizable monomers: divinyl-benzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, and methyl acrylate.

48. The method of claim 46 or 47, wherein said sorbent comprises solid forms comprising particles having a diameter in the range of from about 0.1 microns to about 200 microns; and are characterized as having a pore structure having a total volume of pore sizes in the range of from 10 Å to 10,000 Å is greater than 0.5 cc/g to 3.0 cc/g dry polymer; wherein the ratio of pore volume between 10 Å to 3,000 Å in diameter to pore volume between 500 Å to 3,000 Å in diameter of the said cross-linked polymeric material is smaller than 7:1 and wherein the ratio of pore volume between 10 Å to 3,000 Å in diameter to pore volume between 10 Å to 6,000 Å in diameter of said cross-linked polymeric material is less than 2:1.

49. The method of claim 46 wherein antibodies are attached to the metallic nanoparticles.

50. The method of any of claims 46-49, wherein the sorbent acts ex vivo.

51. The method of any of claims 46-50, wherein the method is part of an extra corporeal treatment.

52. The method of any of claims 46-51, wherein the thrombi is viral-induced thrombi.

53. The method of any of claims 46-51, wherein the thrombi is bacteria-induced thrombi.

54. The method of claim 46, wherein the thrombi have a size in a range of from about 0.5 μm to about 100 μm.

55. The method of claim 46, wherein the thrombi have a size in a range of from about 1 μm to about 90 μm.

56. The method of claim 46, wherein the thrombi have a size in a range of from about 5 μm to about 80 μm.

57. The method of claim 46, wherein the thrombi have a size in a range of from about 10 μm to about 70 μm.

58. The method of claim 46, wherein the thrombi have a size in a range of from about 15 μm to about 65 μm.

59. The method of claim 46, wherein the thrombi have a size in a range of from about 20 μm to about 60 μm.

60. The method of claim 46, wherein the thrombi have a size in a range of from about 30 μm to about 50 μm.

61. The method of claim 46, wherein the thrombi have a size in a range of from about 0.5 μm to about 5 μm; or from about 5 μm to about 10 μm; or from about 10 μm to about 15 μm; or from about 15 μm to about 20 μm; or from about 20 μm to about 25 μm; or from about 25 μm to about 30 μm; or from about 30 μm to about 35 μm; or from about 35 μm to about 40 μm; or from about 40 μm to about 45 μm; or from about 45 μm to about 50 μm; or from about 50 μm to about 55 μm; or from about 55 μm to about 60 μm; or from about 60 μm to about 65 μm; or from about 65 μm to about 70 μm; or from about 70 μm to about 75 μm; or from about 75 μm to about 80 μm; or from about 80 μm to about 85 μm; or from about 85 μm to about 90 μm; or from about 90 μm to about 95 μm; or from about 95 μm to about 100 μm.

62. The method of claim 46, wherein the thrombi have a size in a range of from about 0.5 μm to about 15 μm.

63. The method of claim 46, wherein the thrombi have a size in a range of from about 0.75 μm to about 10 μm.

64. The method of claim 46, wherein the thrombi have a size in a range of from about 1 μm to about 5 μm.

65. The method of claim 46, wherein the thrombi have a size in a range of from about 1.5 μm to about 3 μm.

66. The method of claim 46, wherein the thrombi have a size of about 2 μm.
